(19) United States
(12) Patent Application Publication
(10) Pub. No.: US 2009/0088681 A1
(43) Pub. Date: Apr. 2, 2009

(54) DEVICE AND METHOD FOR THE TREATMENT OF INTRA-ABDOMINAL DISEASE

(76) Inventors: Jon T. McIntyre, Newton, MA (US); Diana Manzanedo, Cambridge, MA (US); Ty Fairneny, Hopkinton, MA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/243,499

(22) Filed: Oct. 1, 2008

Related U.S. Application Data
(60) Provisional application No. 60/976,862, filed on Oct. 2, 2007.

(51) Int. Cl. A61F 7/12; A61M 13/00

(52) U.S. Cl. 604/26; 604/113; 604/506

(57) ABSTRACT

An apparatus, such as a medical device, includes an elongate member having a proximal end portion and a distal end portion. The elongate member defines a lumen extending from the proximal end portion to the distal end portion. The distal end portion of the elongate member is configured to be inserted into a body of a patient. The apparatus includes a heating element configured to heat the lumen defined by the elongate member such that a material within the lumen is heated and can be delivered to bodily tissue.

### CROSS-REFERENCE TO RELATED APPLICATIONS

This application claims priority to U.S. Provisional Application Ser. No. 60/976,862, entitled “Device and Method for the Treatment of Intra-Abdominal Disease,” filed Oct. 2, 2007, which is incorporated herein by reference in its entirety.

### BACKGROUND

The invention relates to treating disease within a body, and more particularly to a system for thermally necrosing diseased tissue within the body and for preventing adhesion to necrosed tissue.

One disease treatable by such a system is endometriosis, a condition affecting millions of women. The condition is characterized by pelvic pain and dysmenorrhea, or painful menstruation, caused when endometrial tissue is present in areas other than the uterus, such as in the peritoneal cavity. Currently, endometriosis is treated with drug therapy, hysterectomy, and/or surgery to remove or coagulate the tissue.

Drug therapy includes administering pain medication and hormonal therapy to interrupt ovarian function and menstrual bleeding. Hormonal therapy may include use of estrogen and progesterone (such as in the form of oral contraceptive pills (OCPs)); progestins; synthetic androgens such as Danazol; Gonadotrophin Releasing Hormone (GnRH) agonists such as goserelin, nafarelin, Buserelin, and leuprorelin; mifepristone; selective progesterone receptor modulators (SPRMs) such as asoprisnil; or aromatase inhibitors. Drug therapy can suppress formation of new endometriosis lesions, but the treatment does not destroy or remove the disease. Drawbacks to drug therapy include problematic side effects. Additionally, many drug therapy treatments can only be administered for a limited time.

Hysterectomy is a treatment involving complete removal of the uterus. This treatment, however, only cures endometriosis some of the time. Furthermore, this treatment is not an option for those women seeking to become pregnant in the future.

The third type of treatment for endometriosis is minimally invasive surgery to remove or coagulate endometriosis lesions. This treatment, which is typically laparoscopic, can include mechanical excision of the tissue, radiofrequency ablation of endometrial lesions, plasma coagulation of endometrial lesions, or vaporization of the tissue by use of carbon dioxide laser. Even using such current surgical intervention-type treatments, however, endometriosis has a high rate of recurrence for a period from six to twenty-four months post-treatment.

Furthermore, adhesion formation may occur at the treatment site in the patient's body following a surgical treatment. Adhesions occur following an inflammatory process, like after a surgical incision, when new fibrous tissue forms and unites tissues normally separated. Resistance to adhesion formation is critical for the first one to two week period post-treatment.

Accordingly, an improved system for treating disease, such as endometriosis, that includes a minimally invasive procedure that thermally necroses the diseased tissue is needed. It is desirable that this improved system combines an improved procedure with direct delivery drug therapy. It is also desirable that this improved system combines an improved procedure with adhesion prevention. An improved medical instrument designed to perform the minimally invasive procedure and direct delivery drug therapy is also needed.

### SUMMARY OF THE INVENTION

An apparatus, such as a medical device, includes an elongate member having a proximal end portion and a distal end portion. The elongate member defines a lumen extending from the proximal end portion to the distal end portion. The distal end portion of the elongate member is configured to be inserted into a body of a patient. The apparatus includes a heating element configured to heat the lumen defined by the elongate member such that a material within the lumen is heated and can be delivered to bodily tissue.

### BRIEF DESCRIPTION OF THE DRAWINGS

FIG. 1 is a schematic illustration of an apparatus according to an embodiment of the invention.

FIG. 2 is a schematic illustration of an apparatus according to an embodiment of the invention.

FIGS. 3 and 4 are perspective views of an apparatus according to an embodiment of the invention.

FIG. 5 is a perspective and partial cross-sectional view of a surgical procedure employing an apparatus according to an embodiment of the invention.

### DETAILED DESCRIPTION OF THE INVENTION

Generally, an embodiment of the invention is an apparatus, such as a medical device, configured to receive a material for necrosing bodily tissue, to heat or melt the material, and to deliver the material through a lumen of the apparatus and onto a target site in or on a body of a patient.

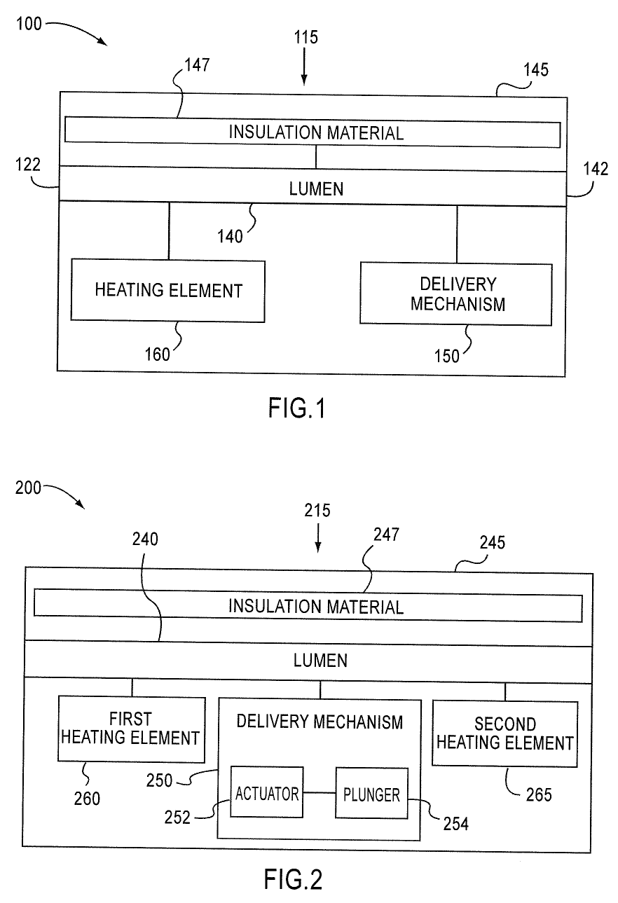

As schematically illustrated in FIG. 1, an apparatus 100 according to one embodiment of the invention includes an elongate member 115, an insulation material 147, a heating element 160, and a delivery mechanism 150. The elongate member 115 defines a lumen 140 extending from a first opening 122 to a second opening 142 of the elongate member.

The elongate member 115 is configured to receive a material (not illustrated in FIG. 1). For example, the elongate member 115 can be configured to receive the material into the lumen 140 via the first opening 122. The heating element 160 is configured to heat the lumen 140 such that the material is heated or melted within the lumen 140. In one embodiment, the heating element 160 is configured to heat the material to a desired temperature. For example, the heating element 160 may be configured to heat the material to an optimal temperature for the material to melt and flow, and then to thermally necrose diseased tissue. Such optimal temperature may, in some cases, be determined by the mass and type of material being inserted into the apparatus 100. The heating element 160 can be any known type of heating mechanism. For example, the heating element 160 can be a heating coil or a heating rod. In some embodiments, an electrical connection is needed to provide power to the heating element 160.

The delivery mechanism 150 is operatively coupled to the elongate member 115. The delivery mechanism 150 is configured to move at least a portion of the material through at least a portion of the lumen 140 of the elongate member 115. In some embodiments, the delivery mechanism 150 is configured to move at least a portion of the material through the lumen 140, out through the second opening 142, and onto a target site in a body of a patient. In some embodiments, the rate of material delivered into the body is in part controlled by the temperature used to heat the material. The more melted (or fluid-like) the material, the lower the viscosity of the material and the easier the material will flow. In one embodiment, the temperature of the heating element 160 is controlled by a thermocouple or similar temperature sensing equipment that is configured to provide feedback control to the heating element.

The insulation material 147 is disposed between an outer surface 145 of the elongate member 115 and the heating element 160. Additionally, in some embodiments, the insulation material 147 is disposed between the outer surface 145 of the elongate member 115 and the lumen 140. The insulation material 147 is configured to help prevent the outer surface 145 of the elongate member 115 from heating to an undesirably high temperature. For example, the insulation material 147 is configured to prevent the outer surface 145 of the elongate member 115 from reaching a temperature that would burn bodily tissue that contacts the outer surface.

The insulation material 147 also helps maintain the material passing through the lumen 140 at a desirable temperature, such as at a temperature capable of necrosing bodily tissue. In other words, the heating element 160 and the insulation material 147 are configured to control the temperature of the material passing through the lumen 140, thus allowing for a predictable thermal response when the material is delivered onto bodily tissue. For example, in one embodiment, the heating element 160 and the insulation material 147 are configured to maintain the material at a temperature that is within 5° C. of a desired temperature. In another embodiment, the heating element 160 and the insulation material 147 are configured to maintain the material at a temperature that is within 2° C. of a desired temperature.

Although the apparatus 100 is illustrated and described herein as including an insulation material 147, in some embodiments, the apparatus is otherwise configured to help prevent the outer surface 145 of the elongate member 115 from heating to an undesirably high temperature. For example, in some embodiments, the apparatus is configured with an air gap or space between the outer surface of the elongate member and the heating element. In such an embodiment, the air gap or space provides a layer of insulation.

As schematically illustrated in FIG. 2, in another embodiment, the heating element 260 is a first heating element, and the apparatus 200 includes a second heating element 265. The second heating element 265 is configured to help hold or maintain at least one of the temperature or melted state of the material as it passes from a first location to a second location within the lumen 240 of the elongate member 215. For example, using the second heating element 265, the material is maintained in a melted state and is prevented from cooling as the material moves through the lumen 240. The second heating element 265 can be configured to maintain the temperature of the material within any required specification. In one embodiment, for example, the second heating element 265 is configured to hold or maintain the temperature of the material at a temperature that is ±5° C. of a desired temperature. The second heating element 265 can be any known heating mechanism. For example, in one embodiment, the second heating element 265 is a heater rod.

The delivery mechanism 250 is operatively coupled to the elongate member 215 and is configured to move at least a portion of the material through at least a portion of the lumen 240. As shown in the embodiment schematically illustrated in FIG. 2, the delivery mechanism 250 includes an actuator 252 and a plunger 254. The plunger 254 is movably coupled to the apparatus 200. For example, in one embodiment, the plunger 254 is configured to extend through at least a portion of the lumen 240 and thereby advance the material through the lumen when the actuator 252 of the delivery mechanism 250 is actuated. Furthermore, although the delivery mechanism is described as having an actuator and plunger, the delivery mechanism can be any known delivery mechanism configured to move the material through the lumen.

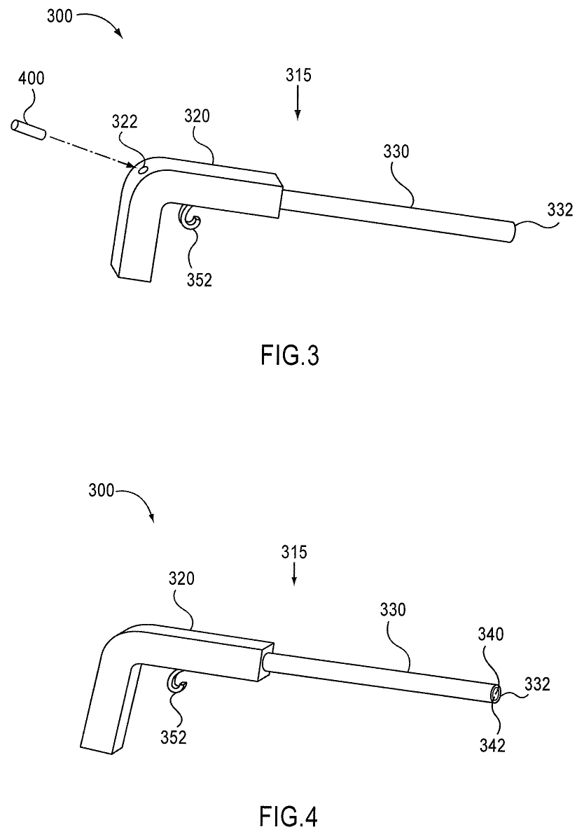

An apparatus 300, such as a medical device, according to one embodiment of the invention is illustrated in FIGS. 3 and 4. In the illustrated embodiment, the apparatus 300 includes an elongate member 315 having a proximal end portion 320 and a distal end portion 330. The elongate member 315 defines a lumen 340 extending from the proximal end portion 320 to the distal end portion 330. Specifically, the lumen 340 extends from a first opening 322 (illustrated in FIG. 3) at the proximal end portion 320 to a second opening 342 (illustrated in FIG. 4) at the distal end portion 330.

The proximal end portion 320 of the elongate member 315 is configured to receive a material 400. In the illustrated embodiment, the proximal end portion 320 is a handle portion configured to receive the material 400 directly into the lumen 340. For example, the proximal end portion 320 can be configured to receive the material 400 into the lumen 340 via the first opening 322. In another embodiment, the apparatus is configured to receive the material 400 in a receiving chamber different than the lumen. For example, in such an embodiment, the receiving chamber may be disposed within the proximal end portion of the elongate member and may be fluidically connected to the lumen.

In some embodiments, the apparatus 300 includes two heating elements. In one such embodiment, the first heating element (not illustrated in FIGS. 3 or 4) is disposed in the proximal end portion 320 of the elongate member 315, and the second heating element (not illustrated in FIGS. 3 or 4) is in the distal end portion 330 of the elongate member. The first heating element is configured to heat the material 400 in the proximal end portion 320 or handle portion of the elongate member 315. For example, the first heating element can be configured to heat the material 400 to the point of melting the material. The second heating element is configured to maintain the temperature of the material 400 as the material is delivered through the lumen 340 from a first location in the proximal end portion 320 to a second location in the distal end portion 330.

As illustrated in FIGS. 3 and 4, in some embodiments, the actuator 352 of the delivery mechanism is disposed on the proximal end portion 320 of the elongate member 315. The plunger (not illustrated in FIGS. 3 or 4) is movably coupled to the elongate member 315 and is configured to extend through at least a portion of the distal end portion 330 of the elongate member 315. The plunger is configured to move the material 400 through at least a portion of the lumen 340.

When the actuator 352 of the delivery mechanism is actuated, the plunger moves the material 400 through at least a portion of the lumen 340. For example, the actuator 352 can cause the plunger to move the material 400 from a first location within the proximal end portion 320 of the elongate member 315 to a second location within the distal end portion 330. Specifically, in one embodiment, the plunger moves at least some of the material 400 through the second opening 342 at a distal tip 332 of the elongate member 315 and onto a target site in the body of the patient.

In one embodiment, the delivery mechanism is configured to deliver a specified amount of material 400 onto the target site in the body of the patient. For example, in one embodiment, the actuator 352 has one or more stops, wherein each stop corresponds to a different degree of extension of the plunger along the lumen 340 of the elongate member 315. The operator can select to move the actuator 352 to one of the one or more stops, and thereby select the degree of extension of the plunger through the lumen 340. Accordingly, the operator of the apparatus 300 can control the amount of material 400 moved through the lumen 340 and delivered onto the target site in the body of the patient.

In the embodiment illustrated in FIGS. 3 and 4, the actuator 352 is a trigger. The actuator 352, however, can be any known mechanism to cause the plunger to move the material 400 through the lumen 340 when the actuator is actuated. For example, in another embodiment, the actuator is a button that may be actuated by depressing the button.

In one embodiment, the delivery mechanism must be actuated to move the material 400 through the lumen 340. In other words, the material 400 moves through the lumen 340 only in response to the actuation of the delivery mechanism. For example, in the embodiment illustrated in FIGS. 3 and 4, the actuator 352 must be squeezed to cause the plunger to move the material 400 through the lumen 340. In another example, a delivery mechanism with an actuator that is a button must be depressed to cause the plunger to move the material 400 through the lumen. Once the trigger or button is released, movement of the material ceases. In another embodiment, the material 400 moves through the lumen 340 independently of a delivery mechanism.

The distal end portion 330 of the elongate member 315 is configured to be inserted into a body of a patient. For example, the distal end portion 330 can be inserted into an insufflated bodily cavity. Specifically, in some embodiments, the distal end portion 330 has a relatively small diameter, which allows the apparatus 300 to be used in minimally invasive medical procedures, including laparoscopic and other procedures. For example, in one embodiment, the outside diameter of the distal end portion 330 is no greater than 10 mm (0.4″) in diameter. In this manner, the distal end portion 330 can fit through a trocar having an inner diameter no greater than 10 mm (0.4″). For example, the distal end portion 330 is configured to fit through a 10 mm (0.4″) trocar. In other embodiments, the outside diameter of the distal end portion 330 is greater than 10 mm (0.4″) in diameter.

In one embodiment, the distal end portion 330 is defined as the portion of the elongate member 315 extending from the distal tip 332 towards the actuator 352 and ending when the outside diameter of the elongate member increases in size. For example, the distal end portion can be the portion of the elongate member 315 extending from the distal tip 332 towards the actuator 352 and ending when the outside diameter of the elongate member is greater than 10 mm (0.4 inches). In another embodiment, the distal end portion 330 is the portion of the elongate member 315 extending from a distal tip 332 of the elongate member 315 to the actuator 352 of the delivery mechanism 350, the actuator being operatively coupled to the proximal end portion 320.

The distal end portion 330 of the elongate member 315 is of a length and configuration to reach a treatment site within a body of a patient. Accordingly, the distal end portion 330 of the elongate member 315 can be inserted into the body of the patient while the proximal end portion 320 remains outside the patient's body. For example, in one embodiment, the distal end portion 330 is no greater than 18″ (46 cm) in length. In another example, the distal end portion 330 is at least 12″ (31 cm) in length, but not greater than 18″ (46 cm) in length. In another embodiment, the distal end portion 330 is at least 18″ (46 cm) in length.

In the embodiment illustrated in FIGS. 3 and 4, the distal end portion 330 of the elongate member 315 is cylindrical. The distal end portion, however, can be any shape. For example, the distal end portion can be a nozzle which tapers as the distal end portion extends from the proximal end portion 320 towards the distal tip 332. In another embodiment, the cross-sectional shape of the distal end portion can be rectangular.

As illustrated in FIG. 3, the proximal end portion 320 of the elongate member 315 is configured to receive a material 400. The material 400 is formulated to be delivered into a body of a patient and to necrose bodily tissue at a target site within the body of the patient. In one embodiment, the material 400 is formulated to necrose bodily tissue when the material is heated to a desired temperature and delivered or applied to the bodily tissue of the patient. For example, when the material 400 is heated to a desired temperature, the heat of the material causes necrosis of the bodily tissue, which occurrence is also referred to as thermally necrosing bodily tissue.

In another embodiment, the material 400 is formulated to superficially necrose bodily tissue when the material is heated to a desired temperature. For example, when the material 400 is heated to a desired temperature and delivered or applied to bodily tissue, only the superficial layers of the bodily tissue are necrosed.

In one embodiment, the material 400 has a low thermal mass, such that the material cools quickly once delivered to a target site within a body of a patient. Thus, the material cools quickly to prevent prolonged exposure to the heat from the material which could otherwise damage healthy bodily materials or tissues proximate to the treatment site. A small amount of material 400 (low thermal mass) may also be used to ensure the material necroses only superficial bodily tissue when delivered to a target site within a body of a patient, and not underlying tissue.

In another embodiment, the material 400 is formulated to prevent adhesion of bodily material to necrosed bodily tissue. For example, the material 400 can be formulated to adhere to necrosed tissue and therein provide a barrier between the necrosed tissue and other bodily material or tissue, thus preventing adhesion. In yet another embodiment, the material is formulated to both thermally necrose bodily tissue when the material is heated and prevent adhesion of bodily material to the necrosed bodily tissue when the material cools.

In some embodiments, the material 400 includes a first material and a second material. For example, in one embodiment, the material includes a first material formulated to necrose bodily tissue and a second material formulated to prevent adhesion formation at the treatment site. In another example, the material includes a first material that is formulated to prevent adhesion formation and a second material that is formulated to enhance the first material's resistance to adhesion formation.

In one embodiment, the material comprises a polymer and a pharmaceutical agent. The pharmaceutical agent can be formulated to elute over a pre-determined period of time. In some embodiments, the pharmaceutical agent is formulated to treat disease. For example, the pharmaceutical agent can be at least one of estrogen and progesterone; progestins; synthetic androgens such as Danazol; GnRH agonists such as goserelin, nafarelin, Buserelin, and leuprorelin; mifepristone; SPRMs such as asoprisnil; or aromatase inhibitors. In some embodiments, the pharmaceutical agent can be formulated to address pain or otherwise provide pain relief. For example, the pharmaceutical agent can be a non-steroidal anti-inflammatory drug (NSAID).

The material 400 can be composed of biodegradable material, which allows the material to be left within a patient's body without requiring later retrieval. For example, the material 400 can be biodegradable material left at the treatment site within the patient's body to prevent adhesion, and then the material can degrade over time as bodily tissue at the treatment site heals.

In one embodiment, the material 400 is a polymer. In some embodiments, the polymer is selected for its melt viscosity, melt temperature, degradation time, or any combination of the foregoing. For example, the material can be a polymer with a desirable degradation time, such as polyhydoxyalkanoate, low molecular weight polycaprolactone, polyglycolic acid, and polylactic-coglycolic acid. In another embodiment, the material can be another polymer, such as polylactic acid, high molecular weight polycaprolactone, poly-3-hydroxybutyrate, and polyglycerol sebacate. In some embodiments, the polymer is biodegradable.

In the embodiment illustrated in FIG. 3, the material 400 may be preformed into a cylindrical shape, like the shape of a pellet or bullet, to be inserted into the first opening 322 at the proximal end portion 320 of the elongate member 315. The material 400 can be preformed into any desired shape configured to be inserted into and melted or heated within the elongate member 315. For example, the material can be preformed into a spherical shape.

The apparatus 300 can be used in a surgical procedure to treat a disease, such as an intra-abdominal disease. In such a procedure, a material formulated to necrose bodily tissue is inserted into a proximal end portion of an apparatus, such as a handle portion of a medical instrument. The material is heated to a desired temperature and is melted within the medical instrument. In one embodiment, the material is melted within the handle portion of the medical instrument. In another embodiment, the material is melted within a lumen of the medical instrument.

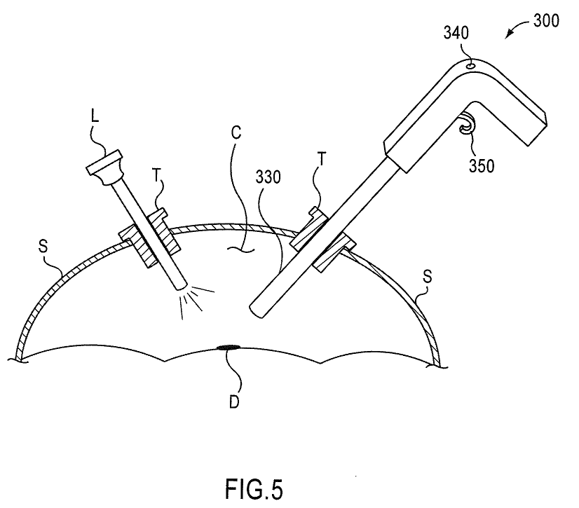

As illustrated schematically in FIG. 5, a body cavity C of a patient may be insufflated. Any known method for insufflating the body cavity C can be used. For example, the body cavity C may be insufflated by introducing a flow of gas into the body cavity, such as introducing carbon dioxide into an abdominal cavity.

The distal end portion 330 of the medical instrument 300 is inserted through skin S and into the body cavity C. In the illustrated embodiment, the distal end portion 330 is inserted into the body cavity C through a trocar T. In another embodiment, the distal end portion 330 can be inserted into the body cavity C without the assistance of a trocar.

The distal end portion 330 of the medical instrument 300 is positioned proximate to the target site D within the body cavity C. Positioning of the distal end portion 330 can be aided by light from light source L and/or a scope (not illustrated), both of which may be inserted into the body cavity C. The light source L in the illustrated embodiment is inserted through the skin S and into the body cavity C through a trocar T. In another embodiment, the light source L is inserted through the skin S and into the body cavity C without the assistance of a trocar.

The actuator 350 of the delivery mechanism is actuated to move the material through the lumen 340 of the medical instrument 300. Accordingly, an amount of the material is delivered to the target site D within the body cavity C. The material treats the bodily tissue at the target site D. Thus, in some embodiments, the material necroses at least a portion of the bodily tissue.

For example, when this procedure is used to treat endometriosis, the distal end portion is positioned proximate to an endometrial lesion and the material is delivered onto the lesion. The lesion is superficially necrosed, while underlying bodily tissue, like the peritoneum, remains undamaged.

In one embodiment, a surgeon or other medical operator can deliver two different materials to the target bodily tissue. For example, a surgeon can first insert a first material for necrosing bodily tissue into the instrument and deliver the material to target bodily tissue, and then insert and deliver a second material for preventing adhesion. In one embodiment, the second material is inserted into the handle or proximal portion of the medical instrument, which insertion can occur while the distal end portion of the elongate member is still inserted into the bodily cavity. The delivery mechanism is actuated to move the second material through the lumen and deliver an amount of the second material to the target site within the body cavity. Using this procedure to treat endometriosis, the surgeon can insert and deliver a first material to necrose the endometrial lesion, and then insert and deliver a second material to prevent adhesion to the necrosed tissue.

In another example, a surgeon can insert and deliver a first material to necrose bodily tissue, and then insert and deliver a second material for addressing pain, like an NSAID, or a second material for treating disease. In treating endometriosis, for example, the surgeon can insert and deliver a first material to necrose the endometrial lesion, and then insert and deliver a pharmaceutical agent, such as those identified above.

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents.

What is claimed is:

1. A medical device, comprising:
an elongate member having a proximal end portion and a distal end portion, the elongate member defining a lumen extending from the proximal end portion to the distal end portion, the distal end portion of the elongate member being configured to be inserted into a body of a patient;
a heating element configured to heat the lumen defined by the elongate member such that a material within the lumen is heated; and
a delivery mechanism operatively coupled to the elongate member, the delivery mechanism configured to move at least a portion of the material through at least a portion of the lumen of the elongate member.

2. The medical device of claim 1, wherein the delivery mechanism is configured to deliver a specified amount of the material through the lumen and onto a target site in the body of the patient.

3. The medical device of claim 1, wherein the delivery mechanism has an actuator and a plunger, the actuator being disposed on the proximal end portion of the elongate member, the plunger being coupled to the actuator, the actuator configured to move the plunger when the actuator is actuated, the plunger configured to move the material through at least a portion of the lumen.

4. The medical device of claim 1, wherein the distal end portion is configured to fit through a trocar having an inner diameter no greater than 10 mm.

5. The medical device of claim 1, further comprising:
insulation material disposed between an outer surface of the elongate member and at least one of the heating element and the lumen.

6. The medical device of claim 1, wherein the heating element is configured to heat the material to a desired temperature.

7. The medical device of claim 1, the heating element being a first heating element, further comprising a second heating element, the second heating element configured to heat the material as it passes from a first location within the lumen to a second location within the lumen.

8. The medical device of claim 7, further comprising:
insulation material disposed between the second heating element and an outer surface of the elongate member.

9. The medical device of claim 1, the heating element being a first heating element, further comprising a second heating element configured to maintain the material at a temperature that is within 5° C. of a desired temperature.

10. The medical device of claim 1, wherein the material is configured to necrose bodily tissue at a target site in the body of the patient when the material is heated to a desired temperature.

11. The medical device of claim 1, wherein the material has a low thermal mass.

12. The medical device of claim 1, wherein the material is a polymer.

13. The medical device of claim 1, wherein the material is at least one polymer selected from the group consisting of polyhydoxyalkanoate, low molecular weight polycaprolactone, polyglycolic acid, polylactic-coglycolic acid, polylactic acid, high molecular weight polycaprolactone, poly-3-hydroxybutyrate, and polyglycerol sebacate.

14. The medical device of claim 1, wherein the material is formulated to inhibit adhesion of bodily material to necrosed bodily tissue.

15. The medical device of claim 1, wherein the material comprises:
a polymer; and
a pharmaceutical agent.

16. The medical device of claim 1, wherein the material includes at least one pharmaceutical agent selected from the group consisting of estrogen, progesterone, progestin, synthetic androgen, GnRH agonist, mifepristone; SPRM, aromatase inhibitor, and non-steroidal anti-inflammatory drug.

17. The medical device of claim 1, wherein the material includes a pharmaceutical agent that is formulated to elute over a pre-determined period of time.

18. A method of treating disease, comprising the steps of:
inserting a material formulated to necrose bodily tissue into a proximal portion of a medical instrument;
heating the material within the medical instrument;
inserting a distal end portion of the medical instrument into a bodily cavity; and
delivering an amount of the material to a target site within the bodily cavity.

19. The method of claim 18, wherein heating the material within the medical instrument includes melting the material within the medical instrument.

20. The method of claim 18, further comprising the step of:
necrosing at least a portion of bodily tissue at the target site within the body cavity.

21. The method of claim 18, wherein the distal end portion is inserted into the body cavity through a trocar.

22. The method of claim 18, further comprising the steps of:
inserting a second material into the proximal portion of the medical instrument; and
delivering a desired amount of the second material to the target site within the body cavity.

23. The method of claim 18, further comprising the step of:
actuating a delivery mechanism on the proximal portion of the medical instrument to move the material through a lumen of the medical instrument.

24. The method of claim 18, further comprising the step of:
positioning the distal end portion of the medical instrument proximate to the target site within the body cavity.

25. The method of claim 18, further comprising the step of:
insufflating a body cavity.

26. A medical device, comprising:
an elongate member having a proximal end portion and a distal end portion, the elongate member defining a lumen extending from the proximal end portion to the distal end portion, the distal end portion of the elongate member being configured to be inserted into a body of a patient, the distal end portion of the elongate member being configured to fit through a trocar;
a heating element configured to heat a biodegradable polymer to a desired temperature within the proximal end portion of the lumen defined by the elongate member;
a delivery mechanism operatively coupled to the elongate member, the delivery mechanism configured to move the polymer from a first location in the lumen of the proximal end portion of the elongate member to a second location in the lumen of the distal end portion of the elongate member; and
an insulation material disposed between an outer surface of the elongate member and at least one of the heating element and the lumen.

27. The medical device of claim 26, wherein the biodegradable polymer is formulated to thermally necrose a controlled area of bodily tissue when the polymer is heated to the desired temperature.

28. The medical device of claim 26, wherein the delivery mechanism is configured to deliver a desired amount of polymer onto a target site in the body of the patient.

29. The medical device of claim 26, wherein the heating element is a first heating element, the medical device further comprising:
a second heating element disposed within the distal end portion of the elongate member configured to heat the polymer as it passes from a first location within the lumen to a second location within the lumen.
